(19) United States
(12) Patent Application Publication
(10) Pub. No.: US 2026/0191834 A1
(43) Pub. Date: Jul. 9, 2026

(54) PHARMACEUTICAL COMBINATION FOR THE TREATMENT OF MULTIPLE MYELOMA, MONOCLONAL GAMMOPATHY OF UNDETERMINED SIGNIFICANCE, AND SMOLDERING MULTIPLE MYELOMA

(71) Applicant: Kathleen E. CLARENCE-SMITH, Washington, DC (US)

(72) Inventor: Kathleen E. CLARENCE-SMITH, Washington, DC (US)

(21) Appl. No.: 19/128,196

(22) PCT Filed: Nov. 7, 2023
(86) PCT No.: PCT/US2023/036932

§ 371 Date: May 8, 2025

Related U.S. Application Data
(60) Provisional application No. 63/423,931, filed on Nov. 9, 2022.

(51) Int. Cl. A61K 31/43; A61K 31/424; A61P 35/00

(52) U.S. Cl. CPC A61K 31/43; A61K 31/424; A61P 35/00

(57) ABSTRACT

A pharmaceutical combination includes a beta-lactam antibiotic alone or in combination with a beta-lactamase inhibitor useful for treating multiple myeloma, monoclonal gammopathy of undetermined significance, and smoldering multiple myeloma. A method for treating multiple myeloma, monoclonal gammopathy of undetermined significance, or smoldering multiple myeloma includes administering to a patient in need thereof an effective amount of a combination of a beta-lactam antibiotic and a beta-lactamase 2024/102361 inhibitor. Beta-lactam antibiotic for use in combination with a beta-lactamase inhibitor for the treatment of multiple myeloma, monoclonal gammopathy of undetermined significance, or smoldering multiple myeloma in a patient in need of the treatment is also described.

### CROSS-REFERENCE TO RELATED APPLICATIONS

This application claims the benefit of U.S. Provisional Ser. No. 63/423,931 filed Nov. 9, 2022, the disclosure of which is incorporated herein by reference in its entirety.

### FIELD OF THE INVENTION

The present invention pertains to the field of the treatment of Multiple Myeloma, Monoclonal Gammopathy of Undetermined Significance (MGUS), and Smoldering Multiple Myeloma.

### OBJECT OF THE INVENTION

The present invention proposes an improvement in the treatment of Multiple Myeloma by a combination of oral amoxicillin, a penicillin antibiotic, and oral clavulanate potassium ER, a beta-lactamase inhibitor.

The present invention also proposes the following embodiments for use for the treatment of Multiple Myeloma, Monoclonal Gammopathy of Undetermined Significance, and Smoldering Multiple Myeloma:
1. A combination of orally, intravenously, or subcutaneously administered amoxicillin and a beta-lactamase inhibitor;
2. A combination of amoxicillin, a penicillin antibiotic, and clavulanate potassium;
3. A combination of a penicillin antibiotic and a beta-lactamase inhibitor;
4. A combination of orally, intravenously, or subcutaneously administered beta lactam antibiotics and beta lactamase inhibitors.

### DEFINITIONS

Multiple Myeloma (MM): Multiple myeloma is a malignant disease characterized by proliferation of clonal plasma cells in the bone marrow typically accompanied by the secretion of monoclonal immunoglobulins that are detectable in the serum or urine.

Monoclonal Gammopathy of Undetermined Significance (MGUS): Monoclonal Gammopathy of Undetermined Significance is a state that precedes all cases of Multiple Myeloma. Smoldering Multiple Myeloma (SMM): Smoldering Multiple Myeloma is an asymptomatic clonal plasma cell disorder. SMM is distinguished from monoclonal gammopathy of undetermined significance (MGUS) by a much higher risk of progression to multiple myeloma (MM).

Amoxicillin: Amoxicillin is semi-synthetic penicillin antibiotic that became available in 1972. It is available in the US under the brand name AMOXIL®.

Clavulanate potassium: Clavulanate potassium is a beta-lactamase inhibitor, which is extensively used as a combination drug in antibiotic therapy.

Clarithromycin: Clarithromycin a semisynthetic macrolide antibiotic, used for many types of bacterial infections.

Extended Release (ER): Extended Release (or sustained release) of the active ingredient from a composition by any administration route.

### BACKGROUND OF THE INVENTION

Multiple myeloma (MM), a bone marrow-resident hematological malignancy of plasma cells, accounts for slightly more than 10% of all hematologic cancers (Kyle and Rajkumar. Multiple myeloma. Blood. 2008 Mar. 15; 111(6):2962-72.). It has remained largely incurable despite dramatic improvements in patient outcomes thanks to myeloma-targeted and immunomodulatory agents Response to the first course of treatment is usually dramatic; however, relapses are the norm. Multiple Myeloma has gone from being a rapidly fatal disease to become a chronic relapsing remitting disease. Patients ultimately succumb to progressive disease (Minnie S A, Hill G R. Immunotherapy of multiple myeloma. J Clin Invest. 2020 Apr. 1;130(4):1565-1575).

Multiple Myeloma typically occurs after the age of 55 years, with a slight male preponderance. It usually develops from a premalignant condition, referred to as a monoclonal gammopathy of undetermined significance (MGUS), often via an intermediate stage termed smoldering multiple myeloma (SMM; Bianchi G, Anderson K C. Understanding biology to tackle the disease: Multiple myeloma from bench to bedside, and back. CA Cancer J Clin. 2014 November-December; 64(6): 422-44).

Patients typically present with a monoclonal protein in the serum or urine, characteristic bone lesions, increased numbers of immature, abnormal, or atypical plasma cells in the bone marrow, bone pain, weakness, recurrent infections, and often in the weeks that precede diagnosis, a rapid and profound weight loss (Michels T C, Petersen K E. Multiple Myeloma: Diagnosis and Treatment. Am Fam Physician. 2017). The common manifestations of multiple myeloma, such as bone pain, fatigue and weight loss, may be viewed as non-specific and are often initially ignored or missed by patients and medical practitioners (Joshua D E, Bryant C, Dix C, Gibson J, Ho J. Biology and therapy of multiple myeloma. Med J August 2019).

The cause of Multiple Myeloma remains unknown. Genetic sequencing of the myeloma cells'genome has not revealed a specific disease-determining genetic alteration. Multiple disease subclones exist at diagnosis and vary in clinical importance with time and drug sensitivity (Joshua D E, Bryant C, Dix C, Gibson J, Ho J. Biology and therapy of multiple myeloma. Med J August 2019).

It has recently become clear that T cells from Multiple Myeloma patients are able to recognize and eliminate myeloma, although this is subverted in the majority of patients who eventually succumb to progressive disease. T cell exhaustion has been implicated in disease progression (Minnie S A, Hill G R. Immunotherapy of multiple myeloma. J Clin Invest. 2020 Apr. 1; 130(4):1565-1575. doi: 10.1172/JCI 129205. PMID: 32149732; PMCID: PMC 7108923).

In the past 20 years, the treatment for Multiple Myeloma has made great progress. Since the introduction of proteasome inhibitors, immunomodulators, and monoclonal antibodies, the longevity of a patient with multiple myeloma has greatly improved. Although prognosis is improving, Multiple Myeloma remains an incurable disease and most patients will inevitably relapse. (Premkumar V, Bhutani D, Lentzsch S. Modern Treatments and Future Directions for Relapsed/Refractory Multiple Myeloma Patients. Clin Lymphoma Myeloma Leuk. 2020).

Recently, the field of multiple myeloma treatment has entered a new era with antibody-based approaches including antibody-drug conjugates, and bispecific T-cell antibodies, further improving patients'longevity (Hosoya H, Sidana S. Antibody-Based Treatment Approaches in Multiple Myeloma. Curr Hematol Malig Rep. 2021). Several years ago, clarithromycin was reported to have efficacy in the treatment of Multiple Myeloma, lending support to the hypothesis that Multiple Myeloma could be an infectious disease. Clarithromycin is a semisynthetic macrolide antibiotic, used for many types of bacterial infections, including treatment of Lyme disease and eradication of gastric infection with Helicobacter pylori (Van Nuffel A M, Sukhatme V, Pantziarka P, Meheus L, Sukhatme V P, Bouche G. Repurposing Drugs in Oncology (ReDO)-clarithromycin as an anti-cancer agent. Ecancer medical science. 2015 Feb. 24;9:513). Recently, the efficacy of clarithromycin f as an add-on to chemotherapies for treating multiple myeloma has been reported and confirmed. However, given alone as a single treatment, it has no efficacy for Multiple Myeloma, calling into question the possibility that Multiple Myeloma could be an infectious disease. (Takemori N, Ooi HK, Imai G, Hoshino K, Saio M. Possible mechanisms of action of clarithromycin and its clinical application as a repurposing drug for treating multiple myeloma. Ecancer medical science. 2020 Aug. 18;14:1088; Chai K Y, Byrne A L, Morison I M. Roxithromycin monotherapy inducing a partial response in a patient with myeloma: a case report. J Med Case Rep. 2018 May 10; 12(1):124). Recent findings, however, lend renewed support to the hypothesis that Multiple Myeloma could be an infectious disease. Subsets of mature B cell neoplasms are linked to infection with intracellular pathogens such as Epstein-Barr virus (EBV), hepatitis C virus (HCV), or Helicobacter pylori (Su et al., 2019), but the findings are contradictory. Bosseboeuf et al. (2017) investigated whether the monoclonal IgG produced by patients diagnosed with MGUS or multiple myeloma targets infectious pathogens. Antigen specificity of purified IgG from a large patient cohort (n=244) was determined using a multiplex infectious-antigen array (MIAA). The authors showed that in certain subsets of patients, monoclonal IgG in MGUS and in multiple myeloma specifically target certain infectious pathogens (Bosseboeuf A, Feron D, Tallet A. Rossi C, Charlier C, Garderet L, Caillot D, Moreau P, Cardo-Vila M. Pasqualini R, Arap W, Nelson A D, Wilson B S, Perremilt H, Piver E, Weigel P, Girodon F, Harb J, Bigot-Corbel E, Hermouet S. Monoclonal IgG in MGUS and multiple myeloma targets infectious pathogens. JCI Insight. 2017 Oct. 5;2(19):e95367). Thus, asymptomatic persistent infections over several years, leading to T-cell exhaustion, could be at the origin of Multiple Myeloma.

The present invention relates to the use of an antibiotic, namely, the combination amoxicillin-clavulanic acid, for the treatment of Multiple Myeloma.

Amoxicillin is a beta-lactam antibiotic. It is used to treat bacterial infections, such as chest infections (including pneumonia) and dental abscesses. It can also be used together with other antibiotics and medicines to treat stomach ulcers. It's often prescribed for children to treat ear infections and chest infections.

Clavulanic acid blocks beta lactamase, an enzyme produced by bacteria to inactivate antibiotics. This reduces resistance and enhances the activity of antibiotics against bacteria. As a chiral drug, clavulanate potassium is the only effective stereoisomer revealing prominent activities (Li Z, Li L, Zheng Y, Chen C, Sun T. Diagnostic absolute configuration determination of clavulanate potassium: A comprehensive investigation of chiroptical spectroscopies and theoretical calculations. J Pharm Biomed Anal. 2018 Oct. 25;160:351-359. doi: 10.1016/j.jpba.2018.08.010. Epub 2018 Aug. 7. PMID: 30114614).

Amoxicillin-clavulanic acid is often used as an empiric therapy for many of the World Health Organization's Priority Infectious Syndromes in adults and children, leading to extensive consumption. While amoxicillin and clavulanic acid have similar half-lives, clavulanic acid is more protein bound and even less heat stable than amoxicillin, with primarily hepatic metabolism. It is also more strongly associated with gastrointestinal side effects, including Clostridium difficile infection, and, thus, in oral combination formulations, limits the maximum daily dose of amoxicillin that can be given. The first ratio for an amoxicillin-clavulanic acid combination was set at 4:1 due to clavulanic acid's high affinity for β-lactamases; ratios of 2:1, 7:1, 14:1 and 16:1 are currently available in various regions. Comparative effectiveness data for the different ratios are scarce.

### SUMMARY OF THE INVENTION

The present invention provides for the use of an orally, intravenously, or subcutaneously administered pharmaceutical combination of amoxicillin and clavulonate potassium ER for the treatment of Multiple Myeloma, MGUS, and SMM. Unlike clarithromycin, this anti-infectious treatment is effective when given alone. The present invention also provides for a pharmaceutical combination of a penicillin antibiotic with a beta-lactamase inhibitor, or a pharmaceutical combination of orally administered beta lactam antibiotics and of beta lactamase inhibitors for the treatment of Multiple Myeloma, MGUS, and SMM.

The present invention includes a method for treating multiple myeloma, monoclonal gammopathy of undetermined significance, or smoldering multiple myeloma, comprising administering to a patient in need thereof an effective amount of a combination of a beta-lactam antibiotic and a beta-lactamase inhibitor.

Also, the present invention includes beta-lactam antibiotic for use in combination with a beta-lactamase inhibitor for the treatment of multiple myeloma, monoclonal gammopathy of undetermined significance, or smoldering multiple myeloma in a patient in need of the treatment. Further, the present invention includes a pharmaceutical combination comprising a beta-lactam antibiotic and a beta-lactamase inhibitor for use in the treatment of multiple myeloma, monoclonal gammopathy of undetermined significance, or smoldering multiple myeloma.

The present invention is advantageous and provides technical advantages because it provides a solution to the problem of treating multiple myeloma, monoclonal gammopathy of undetermined significance, or smoldering multiple myeloma, including by providing a method for treating multiple myeloma, monoclonal gammopathy of undetermined significance, or smoldering multiple myeloma as described herein, a beta-lactam antibiotic for use in combination with a beta-lactamase inhibitor for the treatment of multiple myeloma, monoclonal gammopathy of undetermined significance, or smoldering multiple myeloma as described herein, and a pharmaceutical combination comprising a beta-lactam antibiotic and a beta-lactamase inhibitor for use in the treatment of multiple myeloma, monoclonal gammopathy of undetermined significance, or smoldering multiple myeloma as described herein.

### DETAILED DESCRIPTION

The present invention provides for an improved and novel treatment for Multiple Myeloma, MGUS, and SMM consisting of a combination of amoxicillin and clavulanate potassium ER.

Amoxicillin comes as a capsule, a tablet, a chewable tablet, a sublingual tablet, and a suspension (liquid) to take by mouth. It is usually taken every 12 hours (twice a day) or every 8 hours (three times a day) with or without food. The dose of amoxicillin can range from 500 mg to 8000 mg per day administered 2 to 4 times per day. The ratio between the dose of amoxicillin or a pharmaceutically acceptable salt or solvate thereof and the dose of clavulonic acid or a pharmaceutically acceptable salt thereof, can range from 1:1 to 20:1. Length of treatment depends on the type of infection. In MM, MGUS, and SMM, the duration of antibiotic treatment with the combination amoxicillin plus clavulanate potassium ER should be several months.

Equivalent doses of other beta-lactam antibiotics alone or in combination with amoxicillin and of beta-lactamase inhibitors can be used.

The present invention includes the following embodiments:
1. A method for treating multiple myeloma, monoclonal gammopathy of undetermined significance, or smoldering multiple myeloma, comprising administering to a patient in need thereof an effective amount of a combination of a beta-lactam antibiotic and a beta-lactamase inhibitor.
2. The method of embodiment 1, wherein the beta-lactam antibiotic is amoxicillin or a pharmaceutically acceptable salt or solvate thereof and the beta-lactamase inhibitor is clavulanic acid or a pharmaceutically acceptable salt thereof.
3. The method of embodiment 1, wherein the beta-lactam antibiotic is amoxicillin and the beta-lactamase inhibitor is clavulanate potassium.
4. The method of embodiment 1, wherein the beta-lactam antibiotic is amoxicillin and the beta-lactamase inhibitor is clavulanate potassium, and the combination is in an extended release formulation.
5. The method of embodiment 4, wherein the amoxicillin is administered in an amount of from 500 mg to 10,000 mg per day and the clavulanate potassium is administered in an amount of 4 mg to 10,000 mg per day.
6. The method of embodiment 5, wherein the amoxicillin is administered in an amount of from 500 mg to 8,000 mg per day and the clavulanate potassium is administered in an amount of 4mg to 10,000mg Per Day.
7. The method of embodiment 6, wherein the amoxicillin is present in an amount of 1,000mg and the clavulanate potassium is present in an amount of 62.5 mg in the extended release formulation.
8. The method of embodiment 7, wherein the combination of amoxicillin and clavulanate potassium is administered 2 to 4 times per day.
9. The method of embodiment 8, wherein the combination of amoxicillin and clavulanate potassium extended release is administered 2 times per day.
10. The method of embodiment 1, wherein the method is a method for treating multiple myeloma.
11. The method of embodiment 1, wherein the method is a method for treating monoclonal gammopathy of undetermined significance.
12. The method of embodiment 1, wherein the method is a method for treating smoldering multiple myeloma.
13. The method of embodiment 2, wherein the method is a method for treating multiple myeloma.
14. The method of embodiment 2, wherein the method is a method for treating monoclonal gammopathy of undetermined significance.
15. The method of embodiment 2, wherein the method is a method for treating smoldering multiple myeloma.
16. The method of embodiment 3, wherein the method is a method for treating multiple myeloma.
17. The method of embodiment 3, wherein the method is a method for treating monoclonal gammopathy of undetermined significance.
18. The method of embodiment 3, wherein the method is a method for treating smoldering multiple myeloma.
19. A beta-lactam antibiotic for use in combination with a beta-lactamase inhibitor for the treatment of multiple myeloma, monoclonal gammopathy of undetermined significance, or smoldering multiple myeloma in a patient in need of the treatment.
20. The beta-lactam antibiotic for use of embodiment 19, characterized in that the beta-lactam antibiotic is amoxicillin or a pharmaceutically acceptable salt or solvate thereof and the beta-lactamase inhibitor is clavulanic acid or a pharmaceutically acceptable salt thereof.
21. The beta-lactam antibiotic for use of embodiment 19, characterized in that the beta-lactam antibiotic is amoxicillin and the beta-lactamase inhibitor is clavulanate potassium.
22. The beta-lactam antibiotic for use of embodiment 19, characterized in that the beta-lactam antibiotic is amoxicillin and the beta-lactamase inhibitor is clavulanate potassium, and the combination is in an extended release formulation.
23. The beta-lactam antibiotic for use of any one of embodiments 19-22, characterized in that the beta-lactam antibiotic is administered in an amount of from 500 mg to 10,000 mg per day and the beta-lactamase inhibitor is administered in an amount of 4 mg to 10,000 mg per day.
24. The beta-lactam antibiotic for use of any one of embodiments 19-22, characterized in that the beta-lactam antibiotic is administered in an amount of from 500 mg to 8,000 mg per day and the beta-lactamase inhibitor is administered in an amount of 4 mg to 10,000 mg per day.
25 The beta-lactam antibiotic for use of embodiment 22, characterized in that the beta-lactam antibiotic is present in an amount of 1,000 mg and the beta-lactamase inhibitor is present in an amount of 62.5 mg in the extended release formulation.
26 The beta-lactam antibiotic for use of any one of embodiments 19-25, characterized in that the combination of the beta-lactam antibiotic and the beta-lactamase inhibitor is administered 2 to 4 times per day.
27. The beta-lactam antibiotic for use of any one of embodiments 19-25, characterized in that the combination of the beta-lactam antibiotic and the beta-lactamase inhibitor is administered 2 times per day.
28. The beta-lactam antibiotic for use of any one of embodiments 19-27, characterized in that the treatment is the treatment of multiple myeloma.
29. The beta-lactam antibiotic for use of any one of embodiments 19-27, characterized in that the treatment is the treatment of monoclonal gammopathy of undetermined significance.
30. The beta-lactam antibiotic for use of any one of embodiments 19-27, characterized in that the treatment is the treatment of smoldering multiple myeloma.
31. A pharmaceutical combination comprising a beta-lactam antibiotic and a beta-lactamase inhibitor for use in the treatment of multiple myeloma, monoclonal gammopathy of undetermined significance, or smoldering multiple myeloma.
32. The combination for use of embodiment 31, characterized in that the beta-lactam antibiotic is amoxicillin or a pharmaceutically acceptable salt or solvate thereof and the beta-lactamase inhibitor is clavulanic acid or a pharmaceutically acceptable salt thereof.
33. The combination for use of embodiment 31, characterized in that the beta-lactam antibiotic is amoxicillin and the beta-lactamase inhibitor is clavulanate potassium.
34. A method for treating multiple myeloma, monoclonal gammopathy of undetermined significance, or smoldering multiple myeloma in a patient, comprising identifying the germ which is causing the multiple myeloma, monoclonal gammopathy of undetermined significance, or smoldering multiple myeloma in the patient, and then administering to the patient an effective amount of an antibiotic against that germ.

The present invention also includes the following embodiments:
(i) Amoxicillin in doses ranging from 500 mg to 10,000 mg per day for use for the treatment of MM, MGUS, and SMM in combination with clavulanate potassium ER in doses ranging from 4 mg to 10,000 mg per day.
(ii) A beta-lactam antibiotic in daily doses equivalent to embodiment (i) in combination with clavulanate potassium ER in doses ranging from 4 mg to 10,000 mg per day for use for the treatment of MM, MGUS, and SMM.
(iii) A beta-lactam antibiotic in daily doses equivalent to embodiment (i) in combination a beta-lactamase inhibitor in doses equivalent to embodiment (i) for use for the treatment of MM, MGUS, and SMM.
(iv) A beta-lactam antibiotic in daily doses equivalent to embodiment (i) for the treatment of MM, MGUS, SMM.
(v) A beta-lactamase inhibitor in daily doses equivalent to embodiment (i) for the treatment of MM, MGUS, SMM.

The present invention also contemplates the use of ampicillin, a penicillin antibiotic, as a beta-lactam antibiotic, including in combination with clavulanate potassium in particular.

While not being bound by the following hypothesis, the present inventor believes that multiple myeloma, monoclonal gammopathy of undetermined significance, and smoldering multiple myeloma can be caused by germs (in other words, are infectious diseases), and particularly can be caused by many different germs (a multiple germs hypothesis). The paper by Bosseboeuf et al. mentioned above describes a way of identifying the germs against which the monoclonal antibodies of multiple myeloma are directed. The present inventor's concept is that treating those germs with the proper antibiotic will cure multiple myeloma. For more than 40 years, multiple myeloma was suspected to be an infectious disease, and an antibiotic such a clarithromycin had been effective in some cases. The mistake was to think that only one germ could cause multiple myeloma, and since clarithromycin only worked in a few cases, the infectious hypothesis was abandoned. The present inventor is convinced that many different germs can cause multiple myeloma.

### The Formulations

The pharmaceutical compositions of the present invention are formulated with the classic excipients suitable for different ways of administration. Particularly advantageous are the formulations in the form of tablets, multi-score tablets, coated tablets, orally disintegrating tablets, extended release tablets, hard or soft capsules, extended-release capsules, patches for transdermal administration, liquid oral solutions, syrups or suspensions in a predetermined unit form, and vials for the intravenous or subcutaneous administration.

The pharmaceutical compositions may be formulated in oral forms such as tablets or gelatin capsules, wherein amoxicillin and clavulanic acid, or both the active ingredients, are in admixture with a carrier or vehicle. The carrier or vehicle may include a diluent, such as cellulose, dextrose, lactose, mannitol, sorbitol or sucrose; a lubricant, such as calcium or magnesium stearate, polyethylene glycol, silica, or talc; and if needed, a binder, such as magnesium aluminum silicate, gelatin, methylcellulose, sodium carboxymethylcellulose, or polyvinylpyrrolidone.

The oral forms may be tablets coated with sucrose or with various polymers.

Alternatively, the tablets can be manufactured by using carriers such as acrylic and methacrylic acid polymers and copolymers; cellulose derivatives such as hydroxypropylethylcellulose; or other appropriate materials. These materials confer a prolonged or delayed activity by progressively releasing a predetermined quantity of amoxicillin (or pharmaceutically acceptable salt or solvate thereof) or clavulanic acid (or pharmaceutically acceptable salt thereof).

The oral formulations can also be in the form of capsules allowing the extended release of amoxicillin (or a pharmaceutically acceptable salt or solvate thereof); of clavulanic acid (or pharmaceutically acceptable salt thereof); or of both the active ingredients.

A fixed-dose combination according to the present invention may be a dosage unit form consisting of a capsule comprising, for example, amoxicillin, in an amount of 2000 mg; and clavulanate potassium ER, in an amount of 70 mg, in admixture with a pharmaceutical carrier.

For the intended use in the treatment of MM, MGUS, and SMM, amoxicillin in combination with clavulanate potassium is formulated in a pharmaceutical composition, wherein both active ingredients are in admixture with a pharmaceutical carrier.

The dosage, i.e., the amount of active ingredient in a single dose (amount per unit form) to be administered to a patient, can vary widely depending on the age, weight, and the health condition of the patient. This dosage includes the administration of an amount of amoxicillin ranging from 500 mg to 6000 mg, according to the age of the patient, and an effective amount of clavulanic acid equivalent to from 1 mg to 1000 mg, according to the age of the patient, once to 4 times per day.

The above pharmaceutical compositions are formulated in admixture with a pharmaceutical carrier or vehicle for any administration route. For example, the pharmaceutical compositions are in a pharmaceutical dosage unit form for oral, intranasal, transdermal, or rectal administration.

These unit forms are manufactured according to conventional technologies. Particularly advantageous are the formulations in the form of tablets, multi-score tablets, multi-layer tablets, coated tablets, orally disintegrating tablets, extended release tablets, hard or soft capsules, multi-compartment capsules, extended-release capsules, suppositories for rectal administration, patches for transdermal administration, liquid oral solutions, syrups or suspensions in a predetermined unit form, apparatus for intravenous infusion, and vials for the intravenous or subcutaneous administration.

The pharmaceutical compositions may be formulated in oral unit forms such as tablets or gelatin capsules wherein each of the active ingredients are in admixture with a carrier or vehicle that may include a diluent, such as cellulose, dextrose, lactose, mannitol, sorbitol or sucrose; a lubricant, such as calcium or magnesium stearate, polyethylene glycol, silica, or talc; and if needed, a binder, such as magnesium aluminum silicate, gelatin, methylcellulose, sodium carboxymethylcellulose, or polyvinylpyrrolidone.

The oral unit forms may be tablets coated with sucrose or with various polymers for an immediate release. Alternatively, the tablets can be manufactured by using carriers such as acrylic and methacrylic acid polymers and copolymers; cellulose derivatives such as hydroxypropylethylcellulose; or other appropriate materials having a prolonged or delayed activity by progressively releasing a predetermined quantity of active ingredient.

Syrups and orally dispersible tablets may also comprise sweeteners, lubricants, taste-masking agents, binders, and coloring agents.

Unit forms may be formulated in tablets in which Component (a) and Component (b) are each in ER-formulation, for example, a beta-lactam antibiotic and a beta-lactamase inhibitor, each in admixture with hydroxypropyl methyl cellulose or in a film-coated microgranule. These unit forms (a) and (b) are destined to be concurrently or sequentially administered to a patient suffering from MM, MGUS, and SMM in combination with an oral unit form such as a tablet or gelatin capsule wherein Component (a) is formulated with a diluent and a lubricant in an IR-formulation, or in a tablet or capsule for extended release.

The following example illustrates the invention, but the invention should not be construed as limited thereto.

### EXAMPLE 1

A 72-year old female, with a 30-year history of MGUS, presented with sudden multiple fractures that occurred over 7 weeks, and included 6 vertebrae, 2 ribs, and a shoulder. She complained of lack of energy and profound fatigue. Vertebral pain was so intense that walking had become impossible. She had weighed 45 kg for several years, but concomitant with the bone fractures, she rapidly, over a few weeks, lost 20% of her body weight. She had been followed up for MGUS on an annual basis for more than 30 years, and her latest follow-up blood draw done 9 months prior to the multiple fractures showed no change in the monoclonal IgG peak suggesting a stable MGUS. By contrast, a blood draw done at the time of the bone fractures showed considerable increase of the monoclonal gammopathy, showing the conversion of MGUS to Multiple Myeloma in less than 9 months. The patient was treated with a proteasome inhibitor and immunomodulatory agents, followed by autologous hematopoietic stem cell transplantation. Following stem cell transplantation, she did not receive continual suppressive (maintenance) therapy. Over the following 4.5 years, she was followed up for a possible Multiple Myeloma relapse every 12 weeks, but blood draws showed the absence of a monoclonal antibody peak that would have indicated a relapse. Four years after the stem cell transplantation, the patient had not relapsed, but had not regained her pre-myeloma weight of 45 kg, and continued to complain of lack of energy and feeling tired. At this point in time, she was treated for a sinus infection with the generic combination of amoxicillin and clavulanate potassium extended release tablets (1000 mg/62.5 mg) (SANDOZ). She received 2 tablets per day at an interval of 8 hours. Three days after she had started the antibiotic treatment, she reported a sudden return of energy, and she reported her energy level as equivalent to her energy prior to the onset of Multiple Myeloma symptoms, but her weight continued to be stably low at 36 kg. She remained on bid amoxicillin and clavulanate potassium extended release (1000 mg/62.5 mg) for several weeks and during this period she rapidly, over about 6 weeks, and very unexpectedly, re-gained weight to her pre-myeloma weight of 45 kg, and her weight subsequently stabilized at 48 kg. She had received multiple COVID-19 vaccinations, but had not formed anti-COVID antibodies. After starting treatment with amoxicillin and clavulanate potassium extended release (1000 mg/62.5 mg), following a booster shot of COVID-19 vaccination, she was found to have circulating anti-COVID antibodies. Taken together the regained weight, the return of energy, and the ability to vaccinate against COVID, she showed a profound improvement and possibly a cure of Multiple Myeloma.

As shown above, the present invention provides technical advantages because it provides a solution to, e.g., the problems of weight loss, lack of energy, and fatigue resulting from multiple myeloma, monoclonal gammopathy of undetermined significance, or smoldering multiple myeloma, including by providing a method for treating multiple myeloma, monoclonal gammopathy of undetermined significance, or smoldering multiple myeloma as described above, a beta-lactam antibiotic for use in combination with a beta-lactamase inhibitor for the treatment of multiple myeloma, monoclonal gammopathy of undetermined significance, or smoldering multiple myeloma as described above, and a pharmaceutical combination comprising a beta-lactam antibiotic and a beta-lactamase inhibitor for use in the treatment of multiple myeloma, monoclonal gammopathy of undetermined significance, or smoldering multiple myeloma as described above.

While the present invention is described by way of the above disclosure including embodiments as set forth above, a person of ordinary skill in the art will understand that modifications may be made without departing from the spirit and scope of the invention as defined in the following claims.

1. A method for treating multiple myeloma, monoclonal gammopathy of undetermined significance, or smoldering multiple myeloma, comprising administering to a patient in need thereof an effective amount of a combination of a beta-lactam antibiotic and a beta-lactamase inhibitor.

2. The method of claim 1, wherein the beta-lactam antibiotic is amoxicillin or a pharmaceutically acceptable salt or solvate thereof and the beta-lactamase inhibitor is clavulanic acid or a pharmaceutically acceptable salt thereof.

3. The method of claim 1, wherein the beta-lactam antibiotic is amoxicillin and the beta-lactamase inhibitor is clavulanate potassium.

4. The method of claim 1, wherein the beta-lactam antibiotic is amoxicillin and the beta-lactamase inhibitor is clavulanate potassium, and the combination is in an extended release formulation.

5. The method of claim 4, wherein the amoxicillin is administered in an amount of from 500 mg to 10,000 mg per day and the clavulanate potassium is administered in an amount of 4 mg to 10,000 mg per day.

6. The method of claim 5, wherein the amoxicillin is administered in an amount of from 500 mg to 8,000 mg per day and the clavulanate potassium is administered in an amount of 4 mg to 10,000 mg per day.

7. The method of claim 6, wherein the amoxicillin is present in an amount of 1,000 mg and the clavulanate potassium is present in an amount of 62.5 mg in the extended release formulation.

8. The method of claim 7, wherein the combination of amoxicillin and clavulanate potassium is administered 2 to 4 times per day.

9. The method of claim 8, wherein the combination of amoxicillin and clavulanate potassium extended release is administered 2 times per day.

10. The method of claim 1, wherein the method is a method for treating multiple myeloma.

11. The method of claim 1, wherein the method is a method for treating monoclonal gammopathy of undetermined significance.

12. The method of claim 1, wherein the method is a method for treating smoldering multiple myeloma.

13. The method of claim 2, wherein the method is a method for treating multiple myeloma.

14. The method of claim 2, wherein the method is a method for treating monoclonal gammopathy of undetermined significance.

15. The method of claim 2, wherein the method is a method for treating smoldering multiple myeloma.

16. The method of claim 3, wherein the method is a method for treating multiple myeloma.

17. The method of claim 3, wherein the method is a method for treating monoclonal gammopathy of undetermined significance.

18. The method of claim 3, wherein the method is a method for treating smoldering multiple myeloma.

19-30. (canceled)

31. A pharmaceutical combination comprising a beta-lactam antibiotic and a beta-lactamase inhibitor for use in the treatment of multiple myeloma, monoclonal gammopathy of undetermined significance, or smoldering multiple myeloma.

32. The combination for use of claim 31, characterized in that the beta-lactam antibiotic is amoxicillin or a pharmaceutically acceptable salt or solvate thereof and the beta-lactamase inhibitor is clavulanic acid or a pharmaceutically acceptable salt thereof.

33. The combination for use of claim 31, characterized in that the beta-lactam antibiotic is amoxicillin and the beta-lactamase inhibitor is clavulanate potassium.

34. A method for treating multiple myeloma, monoclonal gammopathy of undetermined significance, or smoldering multiple myeloma in a patient, comprising identifying a germ which is causing the multiple myeloma, monoclonal gammopathy of undetermined significance, or smoldering multiple myeloma in the patient, and then administering to the patient an effective amount of an antibiotic against that germ.
